(19) United States
(12) Patent Application Publication
(10) Pub. No.: US 2005/0070816 A1
(43) Pub. Date: Mar. 31, 2005

(54) Method of adjusting bladder capacity and voiding frequency measurements

(76) Inventor: Alfred C. Coats, Houston, TX (US)

Correspondence Address: BEN D TOBOR;BRACEWELL & PATTERSON, LLP, POST OFFICE BOX 61389, HOUSTON, TX, 77208-1389, US

(21) Appl. No.: 10/954,929

(22) Filed: Sep. 30, 2004

Related U.S. Application Data
(60) Provisional application No. 60/507,350, filed on Sep. 30, 2003.

(51) Int. Cl. A61B 5/00

(52) U.S. Cl. 600/561; 600/300; 128/920

(57) ABSTRACT

A method for diagnosing a urinary tract abnormality in an individual is provided by measuring the individual's bladder capacity and voiding frequency, adjusting bladder capacity and voiding frequency measurements obtained from a normal population, and comparing the individual's measurements with the adjusted measurements from the normal population. This method removes the influence of total volume voided on these measurements and reduces the range, or variability, of these measurements from the normal population, therefore, improves the diagnostic and clinical utility of these measurements.

### RELATED APPLICATIONS

This patent application claims the benefit of U.S. Provisional Patent Application Ser. No. 60/507,350 filed on Sep. 30, 2003, which is incorporated by reference in its entirety.

### BACKGROUND OF THE INVENTION

1. Field of the Invention

The present invention generally relates to medical diagnosis. More specifically, the present invention relates to a method for diagnosing a urinary tract abnormality in an individual by adjusting bladder capacity and voiding frequency measurements by removing the effect of total volume voided upon these measurements.

2. Description of the Related Art

The voiding diary is a record made by a patient of all of his or her urinations (i.e., voids) over a period of time—usually one to seven days. Typically, the time and volume of each void are recorded. The voiding diary is widely considered among urologists and gynecologists to be a very useful diagnostic tool. Its clinical use, both to diagnose disorders of urination and to assess treatment effectiveness, has grown dramatically in recent years. However, in spite of its widespread clinical use, the voiding diary's diagnostic capability is limited because there is a wide overlap between results from persons with, and without, a lower urinary tract abnormality. For example, researchers have demonstrated an overlap of about 40% of bladder-diary measured voiding frequencies and bladder capacities of asymptomatic women and female patients with incontinence, even though statistical testing demonstrated significantly higher voiding frequencies and lower bladder capacities from the patients.

The two most important clinical measurements obtained from the voiding diary are bladder capacity and voiding frequency. Either the average, or maximum, volume of urine passed in a single void during the voiding diary period may be used as a measurement of bladder capacity. Voiding frequency is typically expressed as the number of voids per 24 hours. It is generally agreed among those skilled in the art that total volume voided is directly related to the total amount of fluid ingested, and therefore, “total volume voided” and “total fluid ingested” will be hereafter used interchangeably.

Currently, caregivers determine the degree of bladder capacity and voiding frequency abnormalities by comparing these measurements from patients with frequency distributions of these measurements from asymptomatic persons (the “reference”, or “normal” population).

The strong influence of total volume voided on bladder capacity and voiding frequency significantly reduces the diagnostic efficacy, if directly comparing bladder capacity and voiding frequencies between abnormal and normal populations. The total amount of fluid ingested, hence total volume voided, varies widely from person to person. This high variability of total volume voided substantially increases the variability of bladder capacity and voiding frequency measurements from the normal population. The increased variability of the normal measurements further decreases the ability of the test to, separate abnormal population from the normal population. Additionally, the influence of total volume voided on voiding frequency and bladder capacity may lead to a false conclusion that a high, or low, measurement of these parameters is caused by an abnormality, when in fact they are simply the result of a high or low total volume voided. For example, a low bladder capacity might be mistakenly attributed to an abnormality of the bladder, when, in fact, it is simply a manifestation of a low amount of total urine voided.

Regardless of the strong influence of total volume voided on bladder capacity and voiding frequency, the medical literature that reports the relationship between total volume voided and bladder capacity, or voiding frequency, does not mention the possibility of adjusting bladder capacity and voiding frequency by total volume voided to improve the diagnostic efficacy of these measurements. One common diagnostic use of the bladder diary is to use decreased bladder capacity and increased voiding frequency as measurements of the severity of urge incontinence—a type of incontinence that is caused by contractions of the bladder wall muscle (i.e., the detrusor) that the patient cannot inhibit (i.e., uninhibited detrusor contractions). This practice assumes that bladder capacity is not affected by total volume voided.

Another common diagnostic application of the voiding diary that ignores the relationship between bladder capacity and total volume voided is the use of the bladder capacity measurement to differentiate stress incontinence (i.e., urinary incontinence caused by coughing, physical exercise, or other activity) from urge incontinence. Patients with stress incontinence tend to have larger bladder capacities than those with urge incontinence. However, adjusting bladder capacity measurement according to total volume voided to improve this distinction has not been suggested.

Bladder capacity and voiding frequency are also widely used to assess the efficacy of treatment of voiding abnormalities such as bladder training regimens and drugs. If, after the treatment, bladder capacity is increased or urination frequency is decreased, the treatment is deemed successful. However, this practice ignores the possible effect of the influence on bladder capacity and voiding frequency of total volume voided and, by implication, of total fluid ingested.

Several published studies report across-subject averages of both total volumes voided and maximum and average voided volumes obtained from bladder diaries. Although, as previously discussed, some of the studies report correlations between total volume voided and voiding frequency or bladder capacity, none of the studies refer to any use of the correlation between total volume voided and bladder capacity or voiding frequency to improve the comparison of a patient's measured bladder capacity with bladder capacity of a normal person.

Therefore, there is clearly a need for a new and effective method of adjusting the bladder capacity and voiding frequency measurement by total volume voided to improve the diagnostic efficacy of these measurements. The present invention fulfills this long-standing need in the art.

### SUMMARY OF THE INVENTION

The present invention includes a method for diagnosing a urinary tract abnormality in an individual. This method may include the steps of measuring the individual's bladder capacity and voiding frequency; adjusting bladder capacity and voiding frequency measurements from a normal population by removing the effect of total volume voided on bladder capacity and voiding frequency; and comparing the individual's bladder capacity and voiding frequency measurements with the adjusted bladder capacity and voiding frequency measurements from the normal population.

The present invention also includes a method for differentiating stress incontinence from urge incontinence in an individual. This method may include the steps of obtaining a plurality of bladder capacity measurements from the individual including total volume voided and bladder capacity; adjusting bladder capacity measurements from a normal population by removing the effect of total volume voided on bladder capacity; and comparing the individual's bladder capacity measurements with the adjusted bladder capacity measurements from the normal population.

The present invention further includes a method for diagnosing a voiding abnormality in an individual. This method may include the steps of measuring the individual's bladder capacity, voiding frequency and total volume voided; adjusting bladder capacity and voiding frequency measurements from a normal population by removing the effect of total volume voided on bladder capacity and voiding frequency; and comparing the individual's bladder capacity and voiding frequency measurements with the adjusted bladder capacity and voiding frequency measurements from the normal population.

The present invention yet further includes a computerized system for diagnosing a urinary tract abnormality in an individual. This system may include a computer having a computer memory for receiving bladder capacity and voiding frequency data from both the individual and a normal population; a first computer memory element stored in the computer memory for adjusting bladder capacity and voiding frequency data from the normal population by removing the effect of total volume voided on bladder capacity and voiding frequency; and a second computer memory element stored in the computer memory for comparing the individual's bladder capacity and voiding frequency data with the adjusted bladder capacity and voiding frequency data from the normal population.

The present invention still yet further includes a computerized system for differentiating stress incontinence from urge incontinence in an individual using bladder capacity measurements. This system may include a computer having a computer memory for receiving bladder capacity data from both the individual and a normal population; a first computer memory element stored in the computer memory for adjusting bladder capacity data from the normal population by removing the effect of total volume voided on bladder capacity; and a second computer memory element stored in the computer memory for comparing the individual's bladder capacity data with the adjusted bladder capacity data from the normal population.

The present invention still yet further includes a computerized system for diagnosing a voiding abnormality in an individual. This system may include a computer having a computer memory for receiving bladder capacity, voiding frequency and total volume voided data from both the individual and a normal population; a first computer memory element stored in the computer memory for adjusting bladder capacity and voiding frequency data from the normal population by removing the effect of total volume voided on bladder capacity and voiding frequency; and a second computer memory element stored in the computer memory for comparing the individual's bladder capacity and voiding frequency data with the adjusted bladder capacity and voiding frequency data from the normal population.

The foregoing described advantages and other advantages of the present invention will be apparent to those skilled in the art, in view of the following detailed description of the preferred embodiment of the present invention, taken in conjunction with the accompanying drawings.

### BRIEF DESCRIPTION OF THE DRAWINGS

The novel features believed characteristic of the invention as well as a preferred mode of use, further objectives, and advantages thereof, will best be understood by reference to the following detailed description of an illustrative embodiment when read in conjunction with the accompanying drawings, wherein:

FIG. 1 is a scatter plot illustrating average volume per void versus total voided volume with data points labeled for demonstration of adjustment of average volume voided in accordance with the present invention;

FIG. 2 is a scatter plot illustrating voiding frequency versus total voided volume with data points labeled for demonstration of adjustment of voiding frequency in accordance with the present invention.

FIG. 3 is bar graph illustrating the effects of total voided volume on average volume voided and voiding frequency (panel A: unadjusted; panel B: adjusted) through the labeled data points as shown in FIGS. 1-2 in accordance with the present invention.

FIG. 4 is a histogram illustrating deviations from fitted curve of average voided volumes (ABSOLUTE VALUES) as shown in FIG. 1 (panel A), and distances of average voided volume versus total voided volume data points from the fitted curve as shown in FIG. 1 (panel B) in accordance with the present invention.

FIG. 5 is a histogram illustrating deviations from fitted curve of voiding frequencies (ABSOLUTE VALUES) as shown in FIG. 2 (Panel A), and deviations of the voiding frequency versus total voided volume data points from the fitted curve as shown in FIG. 2 (panel B) in accordance with the present invention.

FIG. 6 is a plot illustrating average voided volume percentiles from nine patients with urodynamically detected detrusor instability, unadjusted (Absolute Values) and adjusted (Deviation From Fitted Curve) in accordance with the present invention.

FIG. 7 is a plot illustrating maximum voided volume percentiles from nine patients with urodynamically detected detrusor instability, unadjusted (Absolute Values) and adjusted (Deviation From Fitted Curve) in accordance with the present invention.

FIG. 8 is a plot illustrating voiding frequency percentiles from nine patients with urodynamically detected detrusor instability, unadjusted (Absolute Values) and adjusted (Deviation From Fitted Curve) in accordance with the present invention.

While the invention will be described in connection with the preferred embodiments, it will be understood that it is not intended to limit the invention to these embodiments. On the contrary, it is intended to cover all alternatives, modifications, and equivalents, as may be included within the spirit and scope of the invention as defined by the claims hereof.

### DETAILED DESCRIPTION OF THE PREFERRED EMBODIMENTS

Because bladder capacity and voiding frequency are strongly related to total volume voided, the variability of total volume voided significantly increases the variability of the absolute bladder capacity and voiding frequency measurements. The influence of total volume voided on bladder capacity and voiding frequency increases the variability of these parameters by over 50% and over 25%, respectively. Adjusting bladder capacity and voiding frequency so as to remove the effect of total volume voided on these measurements would compensate for the possibility that, for example, small bladder capacity or high voiding frequency might be a manifestation, not of urge incontinence, but simply of a small total volume voided—in the case of small bladder capacity—or high total volume voided—in the case of high voiding frequency. Thus, removing the effect of total volume voided, or total fluid ingested, on bladder capacity and voiding frequency should lead to a more accurate and more sensitive assessment of the effects of urge and stress incontinence on bladder capacity.

The present invention advantageously provides a method of using regression analysis to remove the influence of total volume voided on bladder capacity and voiding frequency. Preferably, the method substitutes for absolute value measurements with distances of voiding frequency and bladder capacity data points from estimating curves, fitted by regression analysis, that characterize the relationship between these variables and total volume voided.

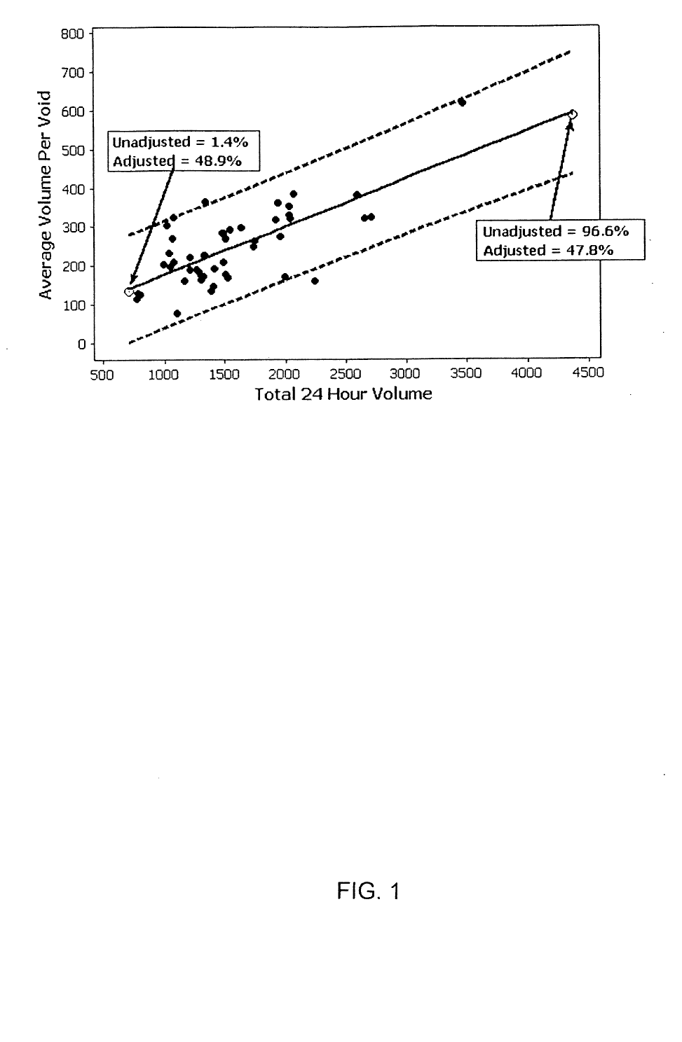

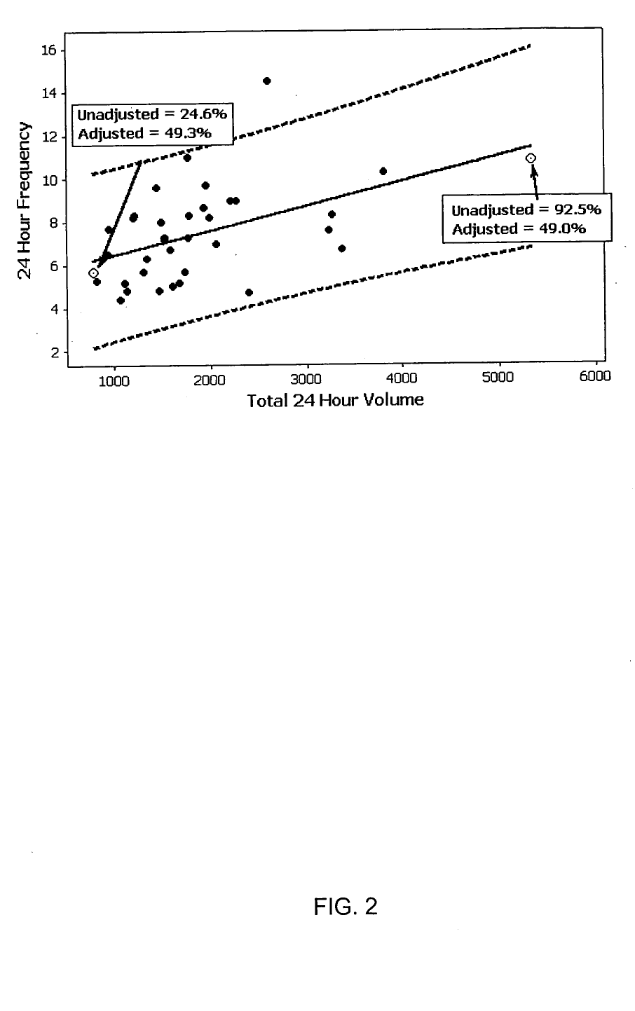

As illustrated in FIG. 1, there exists a strong positive correlation between bladder capacity and total urine volume voided. Data was collected from 50 three-day bladder diaries filled out by volunteers between the ages of 30 and 35 years who had no history of a lower urinary tract abnormality. In addition, as illustrated in FIG. 2, there also exists a weaker, but still significant, positive correlation between voiding frequency and total urine volume voided. Data was collected from 38 three-day bladder diaries filled out by volunteers between the ages of 70 and 78 years who had no history of a lower urinary tract abnormality. In each of FIGS. 1 and 2, the dashed curves above and below the fitted curve represent upper and lower 95 percent probability limits, i.e., the range within which 95% of the data points fall. The scatter plots illustrate correlations of voiding frequency and average voided volume in widely divergent age ranges. Aging produces well-known changes in voiding behavior. For example, voiding frequency tends to increase and bladder capacity tends to decrease with aging. It was found that strong positive correlations across the entire adult age range between bladder capacity—both maximum and average voided volumes—and total volume voided, and weaker, but still highly significant correlations between voiding frequency and total volume voided.

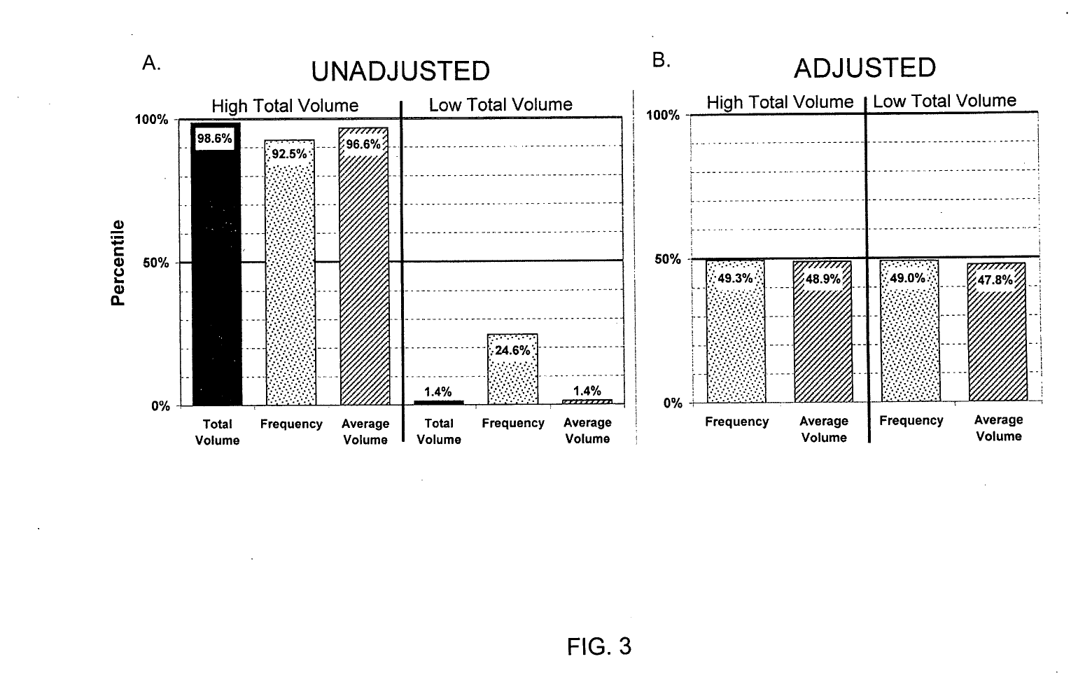

The effect of total volume voided on bladder capacity and voiding frequency can lead to diagnostic inaccuracies. For example, without taking into account the relationship between total voided volume and bladder capacity, it cannot be determined whether a low bladder capacity is a manifestation of a low total volume voided or of a urinary tract abnormality such as detrusor instability, as shown in FIG. 3 with measurements obtained from asymptomatic, or normal, volunteers. Panel A shows unadjusted percentiles into which the two low total volume data points, located on the left of FIGS. 1 and 2, and the two high total volume data points, located on the right of FIGS. 1 and 2, fall. The results suggest that the correlation between total volume and frequency or average volume cause percentiles of the frequency and average volume data points to both vary with total volume. Thus, the frequency and average volume measurements obtained from a subject with high total voided volume are at the upper range, over the 90^{th} percentile, of measurements obtained from the entire population. In contrast, the unadjusted frequency and average volume measurements obtained from a subject with low total volume voided tend to be at the bottom of the range of the entire population. As shown in panel B of FIG. 3, adjusting the data points by calculating the deviation from the fitted curve in accordance with the present invention removes the influence of total volume voided. Since data points close to the estimating curve were selected for this illustration, all of the adjusted measurements are demonstrated to be close to the 50^{th} percentile of the entire asymptomatic population. The average voided volume recorded from the low total volume subject illustrates how failure to adjust the bladder capacity measurement with total volume voided could lead to misdiagnosis. The unadjusted average volume appears very low—at the 1.4^{th} percentile of the entire asymptomatic population as shown in panel A of FIG. 3, whereas adjusted average volume by total volume voided appears to be well within the asymptomatic population range at the 47.8^{th} percentiles, panel B of FIG. 3, for the amount of total volume voided produced by this subject.

Adjusting bladder capacity and voiding frequency by total volume voided also has the advantage of decreasing the overall variability of these measurements in the normal population thus potentially increasing the sensitivity with which these measurements can detect a pathologic condition. The amount of fluid ingested, hence total volume voided, varies widely from person to person. Therefore, as might be expected, total volume voided varies widely from person to person, e.g. from about 540 ml to about 4,300 ml in the present study. The correlations between a widely varying total volume voided and bladder capacity or voiding frequency can account for much of the across-subjects variability of bladder capacity and frequency measurements.

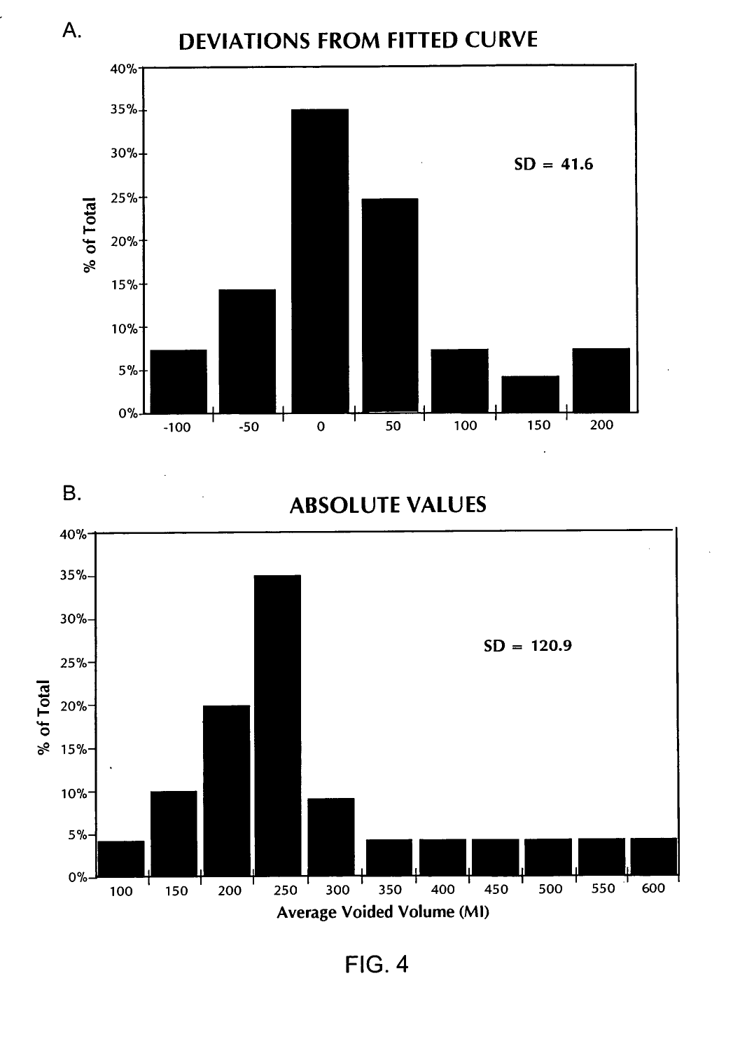

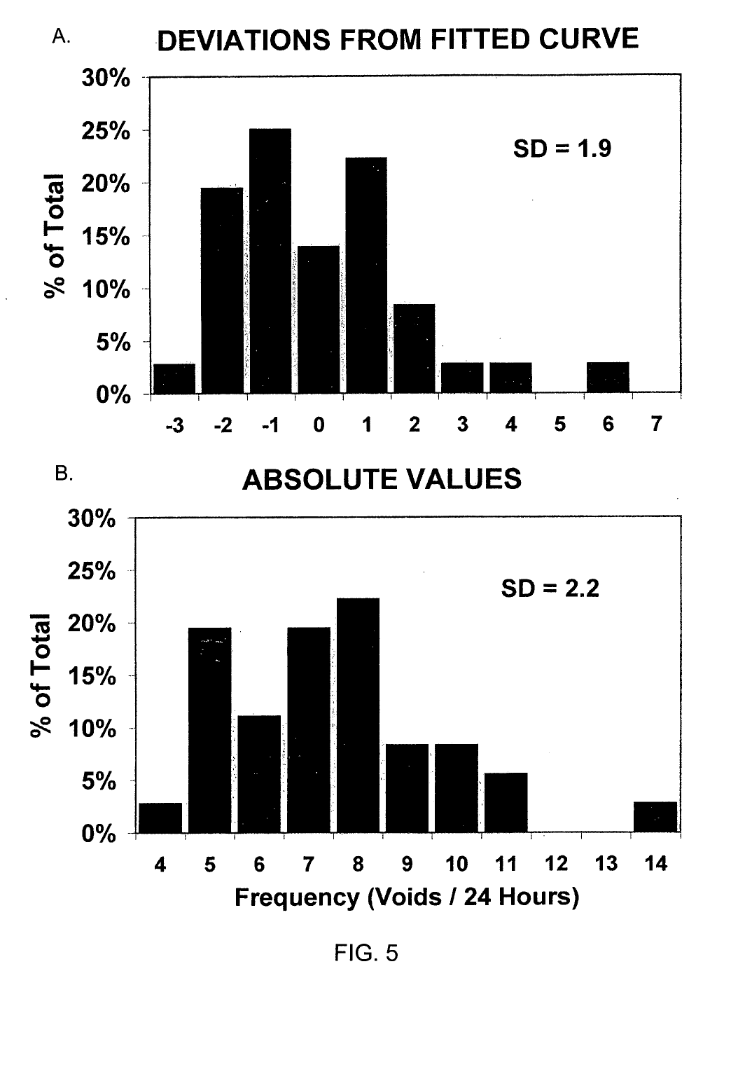

Adjustment of bladder capacity and voiding frequency in accordance with the present invention, removes the contribution of total voided volume variability to the variability of bladder capacity and voiding frequency. That is, expressing bladder capacity and voiding frequency as differences from the fitted curve that estimates their relationship to total voided volume reduces the normal ranges to which a patient's bladder capacity and voiding frequency are compared. The results shown in FIGS. 1, 2, 4 and 5 illustrate the decrease in variability produced by the adjustment. This decreased variability can be appreciated by both the reduced spread of the histogram and the reduced standard deviation (SD), which is a measurement of this spread.

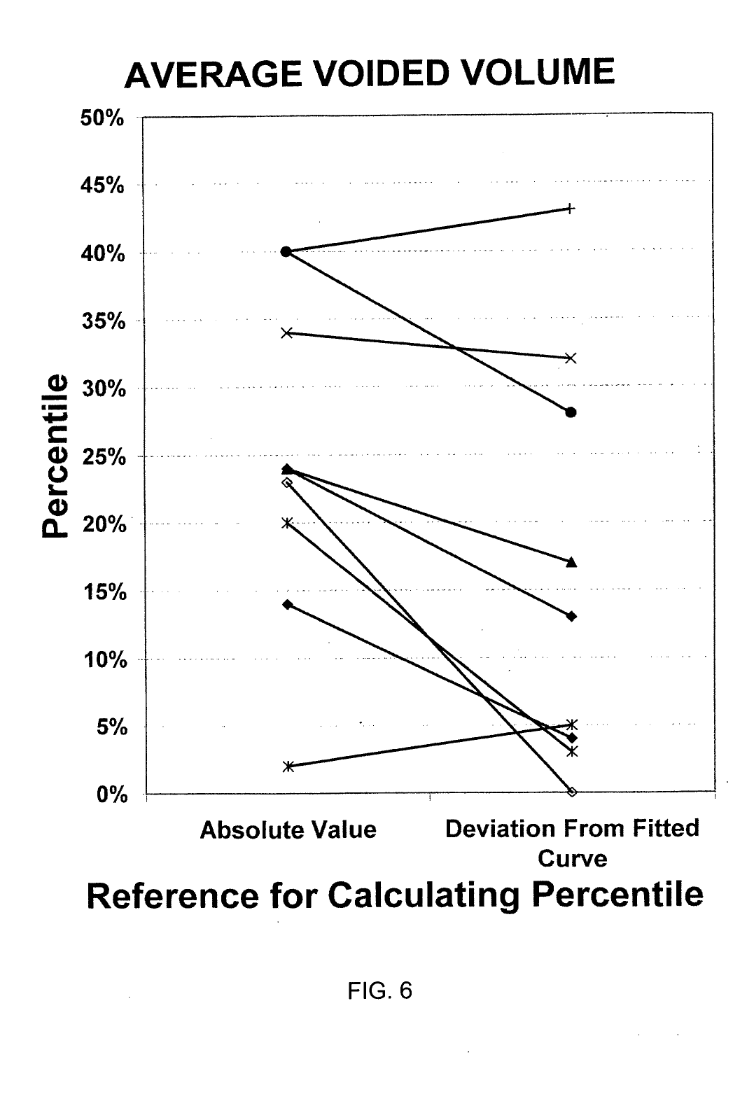

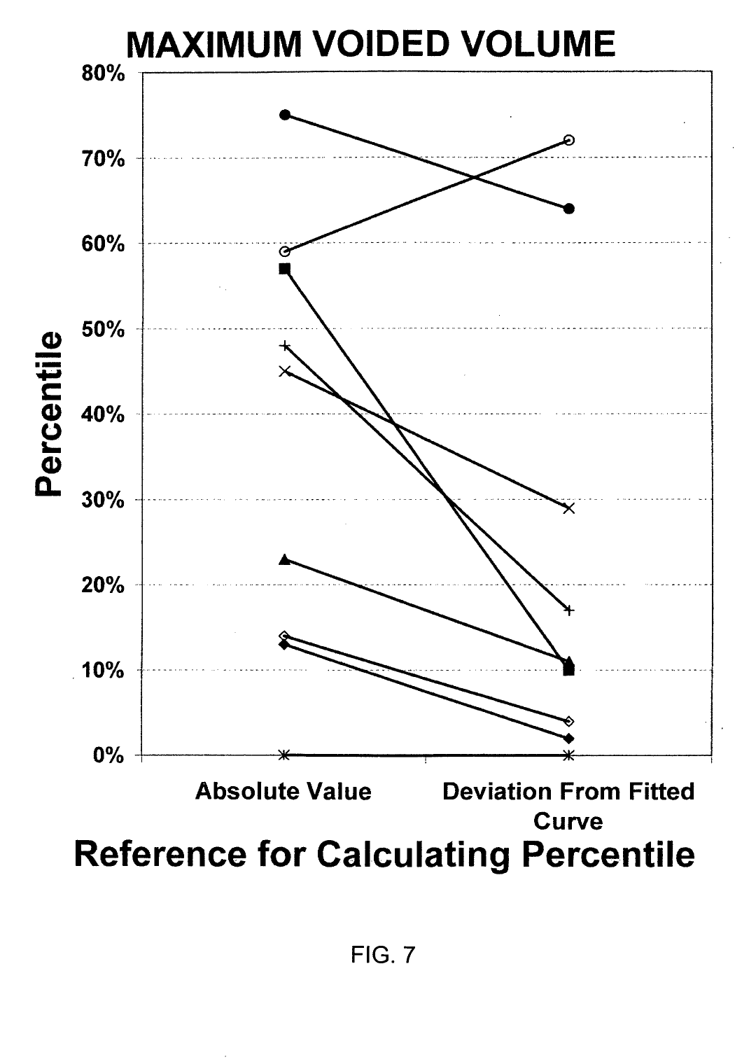

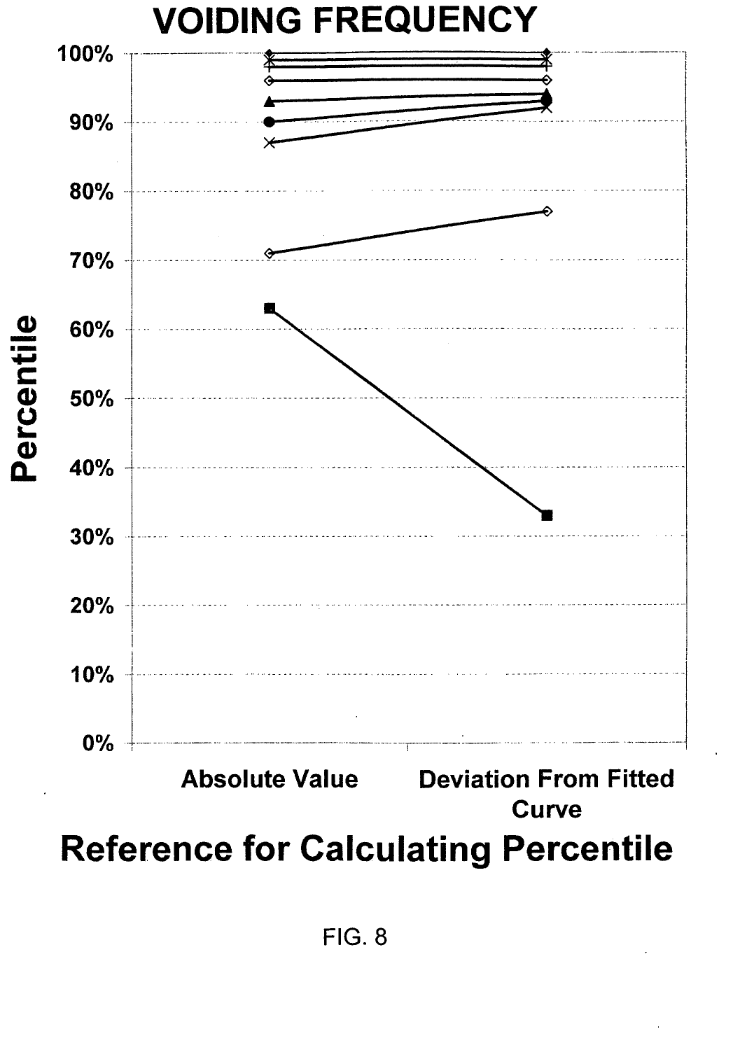

As illustrated in FIGS. 6-8, data collected from nine patients with urodynamically detected detrusor. instability show that the reduced variability of normal voiding frequency and bladder capacity measurements after adjustment better separates patients with known bladder abnormalities from the asymptomatic population. Percentiles were obtained from smoothed cumulative distribution curves of unadjusted (Absolute Value) and adjusted (Deviation From Fitted Curve) measurements. To better appreciate the effect of the adjustment, adjusted and unadjusted percentile data points from each patient are connected with a line. Detrusor instability is known to reduce bladder capacity and increase voiding frequency. Therefore, if the adjustment method of this study increases separation of abnormal from normal population, the percentiles of the adjusted bladder capacity measurements from the detrusor instability patients ought to be less than the percentiles of the unadjusted measurements. Similarly, the adjustment method ought to increase the percentiles of the frequency measurements. FIGS. 6 and 7 demonstrate a readily apparent tendency of the adjustment to decrease the percentiles of both average and maximum voided volumes. However, an increased tendency of the voiding frequency percentiles after adjustment was not observed as shown in FIG. 8. This failure to demonstrate an increase in the adjusted frequency percentiles may have been due to the fact that almost all of the adjusted frequencies were close the available range's maximum, hence had little room to increase further. The clinical data presented in FIGS. 6-8 supports the ability of the adjustment to improve separation of normal from abnormal bladder capacity.

Bladder capacity can also be measured by cystometry, which is a urodynamic test in which the bladder is filled through a urethral catheter. The adjustment method of the present invention may also include adjusting the normal range of cystometric bladder capacity according to total volume voided as measured by the voiding diary in a manner exactly analogous to the above-described adjustment of bladder capacity measured by the voiding diary.

In one embodiment of the present invention, a method for diagnosing a urinary tract abnormality in an individual is taught. This method includes the steps of measuring the individual's bladder capacity and voiding frequency; adjusting bladder capacity and voiding frequency measurements from a normal population by removing the effect of total volume voided on bladder capacity and voiding frequency; and comparing the individual's bladder capacity and voiding frequency measurements with the adjusted bladder capacity and voiding frequency measurements from the normal population.

Preferably, the adjustment is done by using distances from curves fitted by regression analysis, rather than absolute values of the bladder capacity and voiding frequency measurements. The curves fitted by regression analysis advantageously characterize the relationship between the total volume voided and bladder capacity or between the total volume voided and voiding frequency. This adjustment has two beneficial effects. First, it reduces the range, or variability, of bladder capacity and voiding frequency measurements from the normal population. Second, it removes the effect of total volume voided on these measurements, thereby improving the ability of the method to detect the effects of pathology on the measurements.

Still preferably, bladder capacity may be measured by use of a voiding diary or a urodynamic test. This method further diagnoses urge incontinence in the individual by determining decreased bladder capacity, increased voiding frequency, or a combination of decreased bladder capacity and increased voiding frequency in the individual in comparison to the adjusted bladder capacity and voiding frequency measurements from the normal population.

In another embodiment of the present invention, a method for differentiating stress incontinence from urge incontinence in an individual is taught. This method includes the steps of obtaining a plurality of bladder capacity measurements from the individual including total volume voided and bladder capacity; adjusting bladder capacity measurements from a normal population by removing the effect of total volume voided on bladder capacity; and comparing the individual's bladder capacity measurements with the adjusted bladder capacity measurements from the normal population. This method diagnoses stress incontinence in the individual by determining increased or normal bladder capacity in the individual in comparison to the adjusted bladder capacity measurements from the normal population.

In still another embodiment of the present invention, a method for diagnosing a voiding abnormality in an individual is disclosed. This method may include the steps of measuring the individual's bladder capacity, voiding frequency and total volume voided; adjusting bladder capacity and voiding frequency measurements from a normal population by removing the effect of total volume voided on bladder capacity and voiding frequency; and comparing the individual's bladder capacity and voiding frequency measurements with the adjusted bladder capacity and voiding frequency measurements from the normal population. This method diagnoses a voiding abnormality in the individual by determining decreased bladder capacity, increased voiding frequency, or a combination of decreased bladder capacity and increased voiding frequency in the individual in comparison to the adjusted bladder capacity and voiding frequency measurements from the normal population.

In still another embodiment of the present invention, a computerized system for diagnosing a urinary tract abnormality in an individual is taught. This system may include a computer having a computer memory for receiving bladder capacity and voiding frequency data from both the individual and a normal population; a first computer memory element stored in the computer memory for adjusting bladder capacity and voiding frequency data from the normal population by removing the effect of total volume voided on bladder capacity and voiding frequency; and a second computer memory element stored in the computer memory for comparing the individual's bladder capacity and voiding frequency data with the adjusted bladder capacity and voiding frequency data from the normal population.

Preferably, the first computer memory element adjusts the bladder capacity and voiding frequency measurements from the normal population by using distances from curves fitted by regression analysis. This adjustment has two beneficial effects. First, it reduces the range, or variability, of bladder capacity and voiding frequency measurements from the normal population. Second, it removes the effect of total volume voided on these measurements, thereby improving the ability of the method to detect the effects of pathology on the measurements. Still preferably, the second computer memory element further diagnoses a urinary tract abnormality in the individual by determining decreased bladder capacity, increased voiding frequency, or a combination of decreased bladder capacity and increased voiding frequency in the individual in comparison to the adjusted bladder capacity and voiding frequency measurements from the normal population.

In still yet another embodiment of the present invention, a computerized system for differentiating stress incontinence from urge incontinence in an individual using bladder capacity measurements is disclosed. This system may include a computer having a computer memory for receiving bladder capacity data from both the individual and a normal population; a first computer memory element stored in the computer memory for adjusting bladder capacity data from the normal population by removing the effect of total volume voided on bladder capacity; and a second computer memory element stored in the computer memory for comparing the individual's bladder capacity data with the adjusted bladder capacity data from the normal population. This system diagnoses stress incontinence by determining increased or normal bladder capacity in the individual in comparison to the adjusted bladder capacity data from the normal population.

In still yet another embodiment of the present invention, a computerized system for diagnosing a voiding abnormality in an individual is taught. This system may include a computer having a computer memory for receiving bladder capacity, voiding frequency and total volume voided data from both the individual and a normal population; a first computer memory element stored in the computer memory for adjusting bladder capacity and voiding frequency data from the normal population by removing the effect of total volume voided on bladder capacity and voiding frequency; and a second computer memory element stored in the computer memory for comparing the individual's bladder capacity and voiding frequency data with the adjusted bladder capacity and voiding frequency data from the normal population. This system diagnoses a voiding abnormality in the individual by determining decreased bladder capacity, increased voiding frequency, or a combination of decreased bladder capacity and increased voiding frequency in the individual in comparison to the adjusted bladder capacity and voiding frequency data from the normal population.

These methods or computerized systems of the present invention may utilize a number of techniques to adjust bladder capacity and frequency measurements based upon total voided volume. The computerization of the voiding diary makes such adjustments feasible. Specifically, one of the techniques is to plot the patient's data point on a graph of the dependent variable—either bladder capacity or voiding frequency—versus the independent variable—total volume voided—, which includes plots of percentile regions such that the position of the patient's data point relative to the percentile regions indicates the data point's percentile. Scatter plot with the data points removed as presented in FIG. 1 or FIG. 2 could serve as such a nomogram. The dashed lines plot the 95^{th} percentile limits. A patient's data point falling below the lower dashed line would indicate a bladder capacity or voiding frequency below the 95^{th} percentile, hence a measurement that meets the often-used clinical criterion of “abnormal”. A clinician can quickly see from inspection of a nomogram graph where the patient's data point falls in the normal population.

Another technique of adjusting the bladder capacity or voiding frequency measurement based upon total voided volume is to calculate directly the percentile within the normal population of the patient's data point using a formula that incorporates the formula of the fitted curve (sold lines in FIGS. 1 and 2) and the formula of the frequency distribution of the normal data points about the fitted curve. Another technique is to combine the adjustments of these measurements with adjustments of other factors, e.g. age, known to affect bladder capacity and voiding frequency. One way to implement such a combined adjustment would be to calculate the distance between the test data point and the predicted data point yielded by an estimating equation derived from multiple regression of bladder capacity or voiding frequency on total volume voided.

Additional variations of the techniques for implementing the previously described bladder capacity and voiding frequency measurement adjustments according to total voided volume might also be available. For example, alternative techniques other than standard regression might be used to construct the fitted curve. Any number of formulas for the fitted curve, e.g. a straight line, a polynomial or a hyperbola, could be used to fit the total voided volume versus bladder capacity scatter plot. Alternative voiding diary measurements of bladder capacity could be either average voided volume or maximum voided volume. Various mathematical transforms of the data might also be used to remove or minimize nonlinearities in the relationships between variables or systematic variations in amount of scatter about the fitted curves or skewed or leptokurtic or platykurtic shapes of frequency distributions of the residuals about the estimating curve. A formula for a frequency distribution curve fitted to the scatter of the total voided volume versus bladder capacity data points about the fitted curve could be derived from the well-known formula for a normal distribution curve with corrections added for skewness and kurtosis.

Eliminating the influence of total volume voided on bladder capacity and voiding frequency by using distances from curves fitted by regression analysis, rather than absolute values, significantly reduces the range, or variability, of bladder capacity and voiding frequency measurements from normal population, thus increasing the ability of these measurements to distinguish normal from abnormal for more accurate diagnosis. Eliminating the influence of total volume voided on voiding frequency and bladder capacity also increases the ability of the clinician to relate these measurements to clinical abnormalities of the urinary system for more accurate diagnosis. Typically diagnosis is usually made by using the bladder capacity and frequency measurements in conjunction with other clinical data on the patient. The previously described bladder capacity and voiding frequency measurement adjustments according to total volume voided may be used in conjunction with other clinical data known in the art for diagnosing urinary abnormalities.

It is to be understood that the invention is not limited to the exact details of construction, operation, exact materials or embodiment shown and described, as obvious modifications and equivalents will be apparent to one skilled in the art.

1. A method for diagnosing a urinary tract abnormality in an individual, comprising the steps of:
measuring the individual's bladder capacity;
measuring the individual's voiding frequency;
adjusting bladder capacity and voiding frequency measurements from a normal population by removing the effect of total volume voided on bladder capacity and voiding frequency; and
comparing the individual's bladder capacity and voiding frequency measurements with the adjusted bladder capacity and voiding frequency measurements from the normal population.

2. The method of claim 1, wherein the bladder capacity and voiding frequency measurements are adjusted by using distances from curves fitted by regression analysis.

3. The method of claim 2, wherein the curves fitted by regression analysis characterize the relationship between the total volume voided and bladder capacity, or between the total volume voided and voiding frequency.

4. The method of claim 1, wherein the bladder capacity is measured by a voiding diary.

5. The method of claim 1, wherein the bladder capacity is measured by a urodynamic test.

6. The method of claim 1, including the step of diagnosing urge incontinence in the individual by determining decreased bladder capacity, increased voiding frequency, or a combination of decreased bladder capacity and increased voiding frequency in the individual in comparison to the adjusted bladder capacity and voiding frequency measurements from the normal population.

7. A method for differentiating stress incontinence from urge incontinence in an individual using bladder capacity measurements, comprising the steps of:
obtaining bladder capacity measurements from the individual, the measurements including total volume voided, bladder capacity or a combination of both;
adjusting bladder capacity measurements from a normal population by removing the effect of total volume voided on bladder capacity; and
comparing the individual's bladder capacity measurements with the adjusted bladder capacity measurements from the normal population.

8. The method of claim 7, wherein the bladder capacity measurements are adjusted by using distances from curves fitted by regression analysis.

9. The method of claim 8, wherein the curves fitted by regression analysis characterize the relationship between the total volume voided and bladder capacity.

10. The method of claim 7, including the step of diagnosing stress incontinence in the individual by determining increased or normal bladder capacity in the individual in comparison to the adjusted bladder capacity measurements from the normal population.

11. A method for diagnosing a voiding abnormality in an individual, comprising the steps of:
measuring the individual's bladder capacity;
measuring the individual's voiding frequency;
measuring the individual's total volume voided;
adjusting bladder capacity and voiding frequency measurements from a normal population by removing the effect of total volume voided on bladder capacity and voiding frequency; and
comparing the individual's bladder capacity and voiding frequency measurements with the adjusted bladder capacity and voiding frequency measurements from the normal population.

12. The method of claim 11, wherein the bladder capacity and voiding frequency measurements are adjusted by using distances from curves fitted by regression analysis.

13. The method of claim 12, wherein the curves fitted by regression analysis characterize the relationship between the total volume voided and bladder capacity, or between the total volume voided and voiding frequency.

14. The method of claim 11, wherein the bladder capacity is measured by a voiding diary.

15. The method of claim 11, wherein the bladder capacity is measured by a urodynamic test.

16. The method of claim 11, including the step of diagnosing a voiding abnormality in the individual by determining decreased bladder capacity, increased voiding frequency, or a combination of decreased bladder capacity and increased voiding frequency in the individual in comparison to the adjusted bladder capacity and voiding frequency measurements from the normal population.

17. A computerized system for diagnosing a urinary tract abnormality in an individual, comprising:
a computer having a computer memory for receiving bladder capacity and voiding frequency data from both the individual and a normal population;
a first computer memory element stored in the computer memory for adjusting bladder capacity and voiding frequency data from the normal population by removing the effect of total volume voided on bladder capacity and voiding frequency; and
a second computer memory element stored in the computer memory for comparing the individual's bladder capacity and voiding frequency data with the adjusted bladder capacity and voiding frequency data from the normal population.

18. The computerized system of claim 17, wherein the first computer memory element adjusts the bladder capacity and voiding frequency measurements from the normal population by using distances from curves fitted by regression analysis.

19. The computerized system of claim 17, wherein the second computer memory element further diagnoses a urinary tract abnormality in the individual by determining decreased bladder capacity, increased voiding frequency, or a combination of decreased bladder capacity and increased voiding frequency in the individual in comparison to the adjusted bladder capacity and voiding frequency measurements from the normal population.

20. A computerized system for differentiating stress incontinence from urge incontinence in an individual using bladder capacity measurements, comprising:
a computer having a computer memory for receiving bladder capacity data from both the individual and a normal population;
a first computer memory element stored in the computer memory for adjusting bladder capacity data from the normal population by removing the effect of total volume voided on bladder capacity; and
a second computer memory element stored in the computer memory for comparing the individual's bladder capacity data with the adjusted bladder capacity data from the normal population.

21. The computerized system of claim 20, wherein the first computer memory element adjusts the bladder capacity data from the normal population by using distances from curves fitted by regression analysis.

22. The computerized system of claim 20, wherein the second computer memory element further diagnoses stress incontinence by determining increased or normal bladder capacity in the individual in comparison to the adjusted bladder capacity data from the normal population.

23. A computerized system for diagnosing a voiding abnormality in an individual, comprising:
a computer having a computer memory for receiving bladder capacity, voiding frequency and total volume voided data from both the individual and a normal population;
a first computer memory element stored in the computer memory for adjusting bladder capacity and voiding frequency data from the normal population by removing the effect of total volume voided on bladder capacity and voiding frequency; and
a second computer memory element stored in the computer memory for comparing the individual's bladder capacity and voiding frequency data with the adjusted bladder capacity and voiding frequency data from the normal population.

24. The computerized system of claim 23, wherein the first computer memory element adjusts the bladder capacity and voiding frequency data from the normal population by using distances from curves fitted by regression analysis.

25. The computerized system of claim 23, wherein the second computer memory element further diagnoses a voiding abnormality in the individual by determining decreased bladder capacity, increased voiding frequency, or a combination of decreased bladder capacity and increased voiding frequency in the individual in comparison to the adjusted bladder capacity and voiding frequency data from the normal population.
